(19) United States
(12) Patent Application Publication
(10) Pub. No.: US 2008/0035143 A1
(43) Pub. Date: Feb. 14, 2008

(54) Human-powered dry powder inhaler and dry powder inhaler compositions

(76) Inventors: Robert E. Sievers, Boulder, CO (US); Jessica A. Best, Centennial, CO (US); Stephen P. Cape, Boulder, CO (US)

Correspondence Address: DINSMORE & SHOHL, LLP, 1900 CHEMED CENTER, 255 EAST FIFTH STREET, CINCINNATI, OH, 45202, US

(21) Appl. No.: 11/893,040

(22) Filed: Aug. 14, 2007

Related U.S. Application Data
(60) Provisional application No. 60/837,512, filed on Aug. 14, 2006.
(60) Provisional application No. 60/917,045, filed on May 9, 2007.

(51) Int. Cl. A61M 15/00; A61K 9/127; A61M 13/00

(52) U.S. Cl. 128/203.12; 424/450; 604/58

(57) ABSTRACT

In one embodiment, a human-powered dry powder inhaler comprises a human-powered compressible component operable to discharge an air pulse at an outlet at a pressure of about 1-40 psi; an inflatable reservoir operable to receive an air pulse discharged from the human-powered compressible component to provide an aerosol of a dry powder pharmaceutical formulation in the reservoir, the reservoir including an outlet valve; and a receiving mask in communication with the outlet valve and operable to receive an aerosol of dry powder from the reservoir and to deliver the aerosol to at least a mouth or nose of a patient. In another embodiment, the inhaler comprises a human-powered compressible component operable to discharge an air pulse at an outlet of a polymeric pressure release valve at a pressure of about 1-40 psi; and a receiving mask in communication with the outlet of the compressible component and operable to deliver an aerosol of dry powder to at least a mouth or nose of a patient. Methods for delivery of a dry powder pharmaceutical formulation to a patient are conducted in the absence of electrical power and circuitry and pre-pressurized propellant gas. Suitable dry powder pharmaceutical formulations may include myo-inositol and/or maltodextrin as a carrier and active ingredients such as vaccines or siRNA.

### RELATED APPLICATIONS

The present application claims the benefit under 35 U.S.C. § 119 of U.S. Patent Application Ser. Nos. 60/837,512 filed Aug. 14, 2006 and 60/917,045 filed May 10, 2007.

### FIELD OF THE INVENTION

The present invention is directed to dry powder inhalers and to methods of delivering a dry powder pharmaceutical formulation to a patient. The present invention is particularly directed to such inhalers and methods which are human-powered and therefore do not employ electrical power or circuitry or pre-pressurized propellant gases. The present invention is also directed to dry powder pharmaceutical formulations particularly suitable for use in dry powder inhalers.

### BACKGROUND OF THE INVENTION

Dry powder inhalers are well known in the art and are advantageous in various respects to administer pharmaceutical formulations to a patient for nasal or oral delivery to the lungs and other target organs. The Fowler U.S. Pat. No. 2,992,645 discloses a dry powder inhaler which requires a combination of user suction and air pressurized via a squeeze bulb to deliver a medicament or drug. The deBoer et al WO 2004/110538 A1 discloses a flat design for a dry powder inhaler which is used with a peelable blister pack to deliver medicament. Dry powder inhalers driven by propellant gases have also been in use for many years. Recently, there has been an increased focus on developing dry powder inhalers with higher efficiency of delivery. For example, Crowder et al, “An Odyssey in Inhaler Formulation and Design,” Pharmaceutical Technology, July 2001, pp. 99-113, Crowder, “Vibration Technology for Active Dry-Powder Inhalers,” Pharmaceutical Technology, April 200, pp. 52-59, and Hickey et al, “Factors Influencing the Dispersion of Dry Powders as Aerosols,” Pharmaceutical Technology, 18(8): 58-64 (1994), illustrate that the presence of vibration in an inhaler can increase the emitted dose from a dry powder inhaler. In addition, U.S. Pat. Nos. 6,985,798 and 6,971,383 disclose the use of an electrical current to actuate a piezoelectric foil to induce active vibration in the dry powder inhaler, in order to enhance powder delivery. WO 02/053215 discloses a dry powder inhaler having a single dose storage chamber including a seal plate which vibrates to break up a released dose into particles of preferred size.

Other improvements to inhalers are also under development. U.S. Pat. No. 5,823,182 discloses a dry powder inhaler which includes a carrier screen portion which is loaded with a powdered medicament. U.S. Patent Publication No. 2004/0107963 discloses a device and method for deagglomerating powder agglomerates for inhalation. The device includes an inlet connected to a chamber and to a powder source for supplying the chamber with powder agglomerates and a flow of gas that defines a swirling fluid flow inside the chamber. The device also includes an outlet connected to the chamber for inhalation such that the swirling fluid flow in the chamber can exit from the chamber as a longitudinal fluid flow that is directed along a longitudinal axis of the outlet, and a secondary fluid flow that is directed away from the longitudinal axis of the outlet. A mesh in the outlet prevents powder agglomerates above a predetermined size from traversing the mesh, and reduces the secondary fluid flow relative to the longitudinal fluid flow exiting from the chamber to thereby reduce powder deposition in a mouth and throat of a user. U.S. Pat. No. 7,051,734 discloses a medicament respiratory delivery device for delivering a controlled unit dose of an aerosolizable medicament on demand by first pressurizing a pressure chamber in a pressure delivery device upstream of a valve, then opening the valve to open passage sealing membranes having a burst pressure of less than 10 atmospheres and express the medicament through the chamber outlet. Similarly, U.S. Pat. No. 7,040,316 discloses a medicament delivery device including a medicament reservoir and an entrance port and an exit port adjacent the reservoir. A pressurizable gas chamber is disposed adjacent the entrance port, a first frangible membrane extends across the entrance port and separates the reservoir from the gas chamber, and a second frangible membrane extends across the exit port. At least one of the first and second membranes is responsive to a prescribed pressure in the gas chamber to burst to allow gas to flow through the entrance port and the reservoir and to carry the medicament through the exit port.

Dry powder inhalers are advantageous for delivering stable dry powders of pharmaceutical formulations. However, many dry powder inhalers which are currently available for use are expensive, cumbersome in use, and/or not as effective in delivery of active agent as is desired. Moreover, dry powders are of increasing interest for use in areas where refrigeration of liquid pharmaceutical formulations is inconvenient or impossible. One such area is in the provision of vaccines and other pharmaceutical formulations in poor or developing countries, where refrigeration of the large quantities of various vaccines, necessary, for example, for effective child immunization, is not feasible. Thus, a device is needed which can provide easy and effective delivery of such dry powder pharmaceutical formulations, particularly at low cost to satisfy needs in developing countries and otherwise.

### SUMMARY OF THE INVENTION

The present invention provides dry powder inhalers and methods for delivery of dry powder pharmaceutical formulations. The dry powder inhalers and methods are easy for use by non-highly skilled personnel, achieve effective delivery of dry powders, and are economical for use in various applications. The present invention is also directed to certain pharmaceutical formulations which are particularly advantageous for use with dry powder inhalers, and more specifically the dry powder inhalers disclosed herein.

More particularly, in one embodiment, the invention is directed to a human-powered dry powder inhaler, comprising a human-powered compressible component operable to discharge an air pulse at an outlet at a pressure of about 1-40 psi; an inflatable reservoir operable to receive an air pulse discharged from the human-powered compressible component to provide an aerosol of a dry powder pharmaceutical formulation in the reservoir, the reservoir including an outlet valve; and a receiving mask in communication with the outlet valve and operable to receive an aerosol of dry powder from the reservoir and to deliver the aerosol to at least a mouth or nose of a patient.

In another embodiment, the invention is directed to a human-powered dry powder inhaler which comprises a human-powered compressible component operable to discharge an air pulse at an outlet of a polymeric pressure release valve at a pressure of about 1-40 psi; and a receiving mask in communication with the outlet of the compressible component and operable to deliver an aerosol of dry powder to at least a mouth or nose of a patient.

In a further embodiment, the invention is directed to a method for delivery of a dry powder pharmaceutical formulation to a patient, comprising generating an air pulse at a pressure of about 1-40 psi using human power, using the air pulse to provide an aerosol of a dry powder pharmaceutical formulation in an inflatable reservoir, and delivering the resulting aerosol of dry powder pharmaceutical formulation to a receiving mask in communication with at least a mouth or nose of a patient, in the absence of electrical power and circuitry and pre-pressurized propellant gases.

In a further embodiment, the invention is directed to a method for delivery of a dry powder pharmaceutical formulation to a patient, comprising generating an air pulse at an outlet of a polymeric pressure release valve at a pressure of about 1-40 psi using human power, using the air pulse to aerosolize a dry powder pharmaceutical formulation, and delivering the resulting aerosol of dry powder pharmaceutical formulation to a receiving mask in communication with at least a mouth or nose of a patient, in the absence of electrical power and circuitry and pre-pressurized propellant gases.

In a further embodiment, the invention is directed to a method for delivery of a dry powder pharmaceutical formulation to a patient, comprising generating an air pulse at a pressure of about 1-40 psi using human power, using the air pulse to aerosolize a dry powder pharmaceutical formulation, and delivering the resulting aerosol of dry powder pharmaceutical formulation to a receiving mask in communication with at least a mouth or nose of a patient, in the absence of electrical power and circuitry and pre-pressurized propellant gases, wherein the dry powder pharmaceutical formulation comprises an active ingredient and a carrier, wherein the carrier comprises myo-inositol and/or maltodextrin and the dry powder pharmaceutical formulation comprises not more than about 5 weight percent water.

In a further embodiment, the invention is directed to a dry powder pharmaceutical formulation comprising an active ingredient and a carrier, wherein the carrier comprises myo-inositol and/or maltodextrin and the dry powder pharmaceutical formulation comprises not more than about 5 weight percent water.

The dry powder inhalers and the methods of the invention are advantageous for use in various applications, particularly in that they are human powered, therefore not requiring any electrical power or circuitry or pressurized propellant. The dry powder inhalers and methods are easy for use by non-highly skilled personnel, achieve effective delivery of dry powders, and are economical for use in various applications and environments. The dry powder pharmaceutical formulation are advantageous for supplying stable and dispersible formulations. Further embodiments and advantages of the dry powder inhalers, methods and pharmaceutical formulations of the invention will be apparent in view of the following detailed description.

### BRIEF DESCRIPTION OF THE DRAWINGS

The following detailed description will be more fully understood in view of the drawing in which:

FIG. 1 shows a schematic diagram of one embodiment of a dry powder inhaler according to the present invention;

FIG. 2 shows an enlarged view of one embodiment of a compressible component outlet valve suitable for use in a dry powder inhaler according to the present invention;

FIG. 3 shows an enlarged view of an embodiment of a sound vibration generator for use in a dry powder inhaler according to the present invention;

FIG. 4A shows a plan view of portion of a dry powder inhaler according to the present invention, FIG. 4B shows a schematic view of a portion of the dry powder inhaler of FIG. 4A, and FIG. 4C shows a detachable inflatable reservoir and mask thereof for use in the inhaler of FIGS. 4A and 4B, with an aerosolized dose being delivered;

FIG. 5 shows a scanning electron microscopy image of a myo-inositol based dry powder formulation;

FIG. 6 shows measles vaccine virus titers for described myo-inositol based dry powder formulations; and

FIGS. 7A and 7B show dry powder formulations of, respectively, microparticles formed from pure siRNA in an aqueous solution and microparticles formed from equal weights of myo-inositol and siRNA in an aqueous solution.

The embodiments set forth in the drawing are illustrative in nature and are not intended to be limiting of the invention defined by the claims. Moreover, individual features of the drawing and the invention will be more fully apparent and understood in view of the detailed description.

### DETAILED DESCRIPTION

The present invention is directed to human-powered dry powder inhalers and to methods for delivery of a dry powder pharmaceutical formulation to a patient. Within the present disclosure, the term “human-powered” means that the inhaler is operated solely by power supplied by a human, for example the patient or an administrator, without the use of electrical power or circuitry and without the use of a pressurized propellant gas as is commonly employed in current commercially available inhalers. Further, within the present disclosure, the term “dry powder” refers to powders which may be aerosolized for delivery to a patient by nasal and/or oral administration, and, in a specific embodiment, for such administration to the lungs. Suitably, such powders will have an aerodynamic diameter (measured as a function of particle weight and velocity) of from about 0.1 to about 100 microns, although other sized powders may be employed. In embodiments wherein oral delivery to the lungs of a patient is desired, powder particles may advantageously be in the range of from about 1 to about 5 microns, while in embodiments wherein nasal delivery is desired, powder particles may advantageously be in the range of from about 10 to about 30 microns.

The dry powders may be formed by any method known in the art. In one embodiment, the dry powders are formed according to the procedures set for in the Sievers et al U.S. Pat. No. 6,630,121, which is incorporated herein by reference, or by the Carbon Dioxide Assisted Nebulization with a Bubble Dryer® (CAN-BD) process available commercially from Aktiv-Dry, Boulder, Colo. Briefly, in the CAN-BD process, a solution or suspension of an active ingredient in acetone, alcohol, or water is mixed intimately with CO₂ at a low pressure of, for example, about 100 bar to form an emulsion. The emulsion is rapidly expanded to atmospheric pressure through a flow restrictor to generate aerosols of microbubbles and microdroplets. The aerosol plume is dried at temperatures of about 50° C. or less as it mixes with pre-warmed nitrogen or air in a drying chamber. Dry fine powders are collected upon exit from the drying chamber.

The pharmaceutical formulations suitable for use in the dry powder inhalers and methods according to the invention may include one or more active pharmaceutical ingredients as desired. Examples include, but are not limited to, surfactants, insulin, amino acids, enzymes, analgesics, anti-cancer agents, antimicrobial agents, viruses, antiviral agents, antifungal pharmaceuticals, antibiotics, nucleotides, DNAs, antisense cDNAs, RNAs, including siRNAs, peptides, proteins, immune suppressants, thrombolytics, anticoagulants, central nervous system stimulants, decongestants, diuretic vasodialators, antipsychotics, neurotransmitters, sedatives, hormones, anesthetics, anti-inflammatories, antioxidants, antihistamines, vitamins, minerals and other physiologically active materials known to the art. In a specific embodiment, the pharmaceutical formulation comprises a vaccine, anti-viral, antibiotic, anti-inflammatory agent or siRNA. In a more specific embodiment, the active ingredient comprises a measles vaccine.

In one embodiment, the dry powder inhaler pharmaceutical formulations according to the present invention comprise an active ingredient and a carrier, wherein the carrier comprises myo-inositol and/or maltodextrin, and the formulations comprise not more than about 5 weight percent water, more specifically not more than about 2 weight percent water or, in additional embodiments, not more than about 1 weight percent water. In yet further embodiments, the dry powder pharmaceutical formulations suitably comprise not more than about 0.5 weight percent water. Dry powder formulations comprising a moisture sensitive active ingredient preferably comprise less than about 0.5 weight percent water.

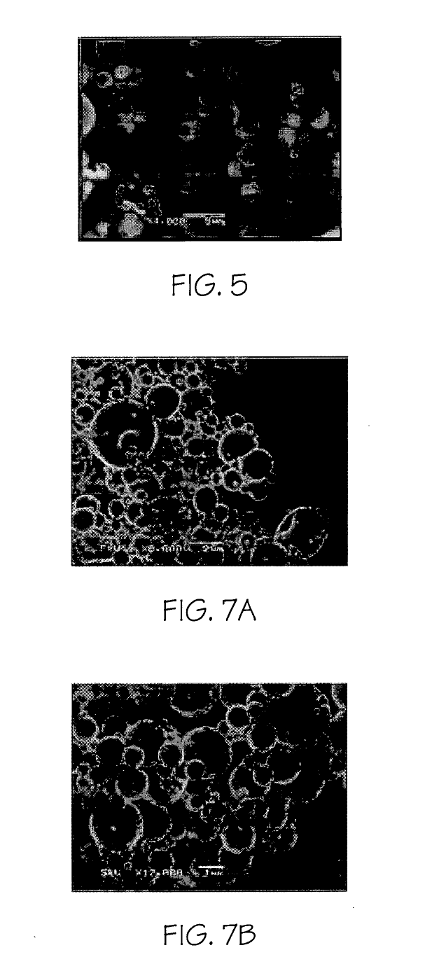

Myo-inositol, also known historically as “meat sugar” or cis-1,2,3,5-trans-4,6-cyclohexanehexyl, is an essential nutrient required by human cells for growth and survival in culture. Free myo-inositol has extremely low toxicity and may be derived from rice. In one embodiment, the pharmaceutical formulation may comprise from about 10 to 100 g/L of myo-inositol or, more specifically, about 50 g/L of myo-inositol. While sorbitol has traditionally been used as a carrier in pharmaceutical formulations such as vaccines, for example in the measles vaccine sold in more than about 100 countries by the Serum Institute of India (SII), sorbitol tends to be sticky and difficult to disperse and tends to pick up water when exposed to moisture. Conversely, the myo-inositol is less hygroscopic than most other sugar excipients and during nebulization and drying with CAN-BD referenced above, nearly spherical particles tend to form, as shown, for example, in FIG. 5. Accordingly, specific embodiments of the dry powder pharmaceutical formulations containing myo-inositol suitably comprise not more than about 1 weight percent water. In yet further embodiments, the dry powder pharmaceutical formulations containing myo-inositol suitably comprise not more than about 0.5 weight percent water.

Maltodextrin is a moderately sweet polysaccharide commonly used as a food additive. It is produced from starch and is usually found as a creamy white hygroscopic powder. Maltodextrin is easily digestible, being absorbed as rapidly as glucose. Maltodextrin can be derived from any starch, for example, corn or potato. In one embodiment, the pharmaceutical formulation may comprise from about 1 to about 40 g/L of maltodextrin or, more specifically, about 20 g/L of maltodextrin. Maltodextrin can improve the dispersibility of a dry powder formulation. Advantageously, maltodextrin does not inactivate live vaccines to the degree seen with materials such as leucine.

In a further embodiment, the dry powder formulations comprise a mixture of myo-inositol and maltodextrin to provide a formulation with improved uniformity and improved powder dispersion stability, particularly at higher relative humidity, thereby allowing an aerosol to be dispersed for a longer period of time. Exemplary mixtures include 25-75 weight percent myo-inositol and 75-25 weight percent maltodextrin, based on a combination of myo-inositol and maltodextrin, although other proportions are acceptable as well. For example, at 70% relative humidity, the dispersibility of a dry powder formulation containing myo-inositol at a level of about 50 g/L can be improved by including about 20 g/L of maltodextrin in the formulation. As a result, the administration time can be increased form about 1 minute to about 5 minutes before the aerosol powder dispersion degradation or instability is significant.

In additional embodiments, the dry powder formulations may include one or more additional excipients or carriers. In one embodiment, the dry powder formulations include a surfactant to render the powder surfaces more lipophilic. One suitable surfactant comprises lecithin, although other surfactants will be apparent to one of ordinary skill in the art.

In a specific embodiment, the dry powder pharmaceutical formulation comprises a vaccine and a myo-inositol carrier, with or without maltodextrin and/or other carriers and excipients. More specifically, the vaccine comprises measles virus. In further embodiments, the dry powder pharmaceutical formulation has a fine particle fraction (FPF) of 50% less than 6 μm (aerodynamic diameter as measured with an Anderson Cascade Impacter), and in some embodiments, a (FPF) of 30% less than 4 μm. The dry powder pharmaceutical formulations containing live virus, for example measles virus, exhibit good activity and good stability. In additional embodiments, the dry powder pharmaceutical formulation retains greater than about 50% activity, more specifically greater than about 70% activity, through processing and/or passes the World Health Organization (WHO) stability test by exhibiting less than 1 log loss of viral activity of the vaccine upon incubation at 37° C. for 7 days.

In another embodiment, the dry powder pharmaceutical formulation according to the invention comprises siRNA or comprises siRNA and myo-inositol. The relative amounts thereof may be varied as desired, but in one embodiment, the dry powder formulation comprises equal weights of siRNA and myo-inositol. In further embodiments, maltodextrin and/or lecithin are included in the siRNA-containing dry powder formulations, with or without myo-inositol. In a specific embodiment, these dry powder formulations are prepared by forming microparticles of siRNA using the CAN-BD as described above, optionally with myo-inositol, maltodextrin, and/or lecithin, and/or other excipients as desired.

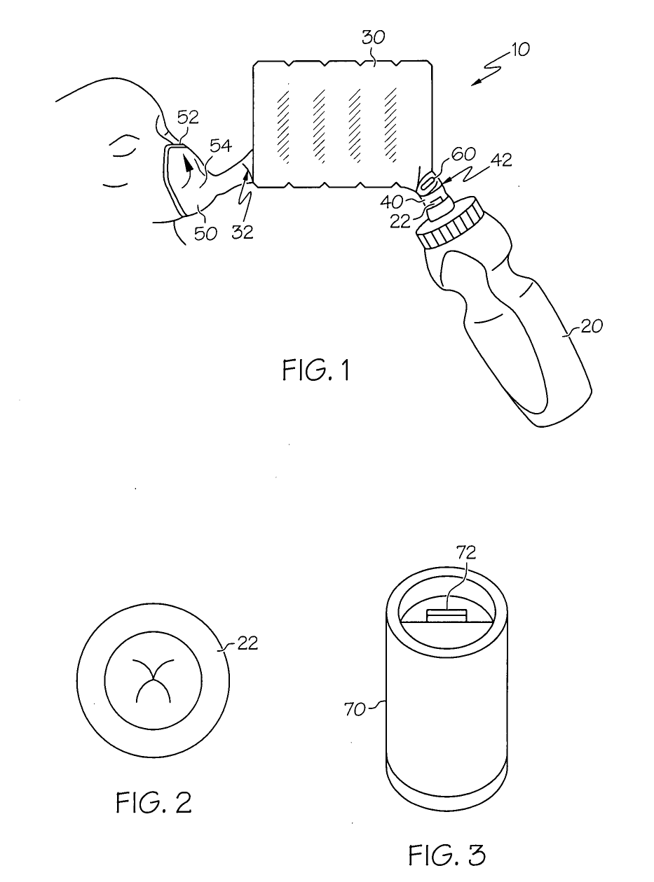

The inhalers according to the present invention are suitable for use with the dry powder pharmaceutical formulations as described herein and for use with other dry powder pharmaceutical formulations as known in the art. FIG. 1 shows a schematic diagram of one embodiment of the human-powered dry powder inhaler 10 according to the invention. The inhaler is suitable for use with uncooperative patients (for example, infants, toddlers, or unconscious individuals) as well as cooperative patients for the prevention or alleviation of disease or injury, or conditions associated therewith. The human-powered dry powder inhaler comprises a human-powered compressible component operable to discharge an air pulse at an outlet at a pressure of about 1-40 psi (gauge), more specifically, at a pressure of about 1-10 psi or at a pressure of about 1-5 psi. In one embodiment, the human-powered compressible component is operable to discharge an air pulse at an outlet at a pressure of about 2 psi. The compressible component is operable to generate the air pulse by slow compression, followed by rapid expansion. For example, the compressible component may comprise a squeezable container, such as, for example, a flexible bottle, balloon, bulb or bag, suitably having a volume of 25 to 1000 mL, fitted with a relatively stiff pressure relief valve which allows for rapid expansion to create the air pulse. This allows human power development of potential energy upon squeezing the bottle, followed by rapid translation to kinetic energy when the valve opens. A pressure reservoir can be charged with compressed air, for example by repetitive pumping by hand or foot with a mechanical pump to generate up to about 100 psi or more to open a pressure relief valve and provide an air pulse at the desired psi.

In FIG. 1, the compressible component 20 is in the form of a plastic squeeze bottle provided with an outlet valve 22, an enlarged view of the inlet side of the outlet valve being provided in FIG. 2. This embodiment of the outlet valve comprises a polymeric pressure relief valve in the form of a four leaf valve. Silicone rubber is a suitable material for forming a polymeric pressure relief valve for use in the inhaler of the invention, although one skilled in the art will appreciate that other polymeric materials may be employed as desired. In an alternative embodiment, the human-powered compressible component may comprise a syringe barrel with a conventional plunger fitted with an outlet valve, for example, a pressure relief valve as shown in FIG. 2. Syringe barrels having a volume of 5 to 500 mL may be suitable in a specific embodiment, although other sized syringes may be suitable in alternate embodiments.

As shown in FIG. 1, the inhaler may optionally further include an inflatable reservoir 30 operable to receive an air pulse discharged from the human-powered compressible component to provide an aerosol of a dry powder pharmaceutical formulation in the reservoir. The inflatable reservoir may suitably be in the form of a collapsed paper or plastic bag in which the aerosol may be temporarily held until inhaled. A transparent inflatable reservoir may be advantageous to allow visual monitoring of the aerosol in the reservoir, for example to confirm formation of the aerosol and that large residues of pharmaceutical formulation do not remain after administration. The reservoir is preferably expandable or contractible with a small pressure change, such as results from tidal breathing. The reservoir may be of any volume as desired. In one embodiment, a volume of approximately 100-300 cm³, more specifically about 200 cm³, is desired. The inflatable reservoir is particularly suitable for use when the inhaler is intended for use with uncooperative patients, for example infants, toddlers, unconscious patients and the like. In certain embodiments, slight pressure may be applied to the inflatable reservoir to assist administration to a patient, but caution should be exercised to avoid any damage to the patient's respiratory tract.

Optionally, a chamber 40 of variable tunable volume that permits throttling of the pulse of air, if necessary, may be provided immediately downstream of the outlet of the compressible component to create a lower pressure air pulse that forms a “softer plume” of aerosol with lower velocity. This chamber may optionally be followed by a softer pressure relief valve 42 to provide a throttled air pulse at an outlet at a pressure of, for example, less than about 2 psi to the inflatable reservoir, or, in embodiments in which the inflatable reservoir is omitted, to a receiving mask 50. In one embodiment, the chamber 40 may be detachable and separately sealed to store and ship an aliquot of a dry powder pharmaceutical formulation. Thus, in one embodiment, the compressible component may be reused with multiple chambers, inflatable reservoirs and receiving masks which are disposed of after one use.

The valves employed in the inhaler are, in one embodiment, suitable one-way valves in order to prevent improper functioning of the device and/or contamination of contents or adjacent atmosphere. Thus, the valve 22 at the outlet of the compressible component can be a one way valve and prevent air flow back towards the compressible component. Similarly, the valve 42 at the outlet of the chamber 40, if employed, can be a one way valve and prevent flow back toward the chamber and only allow flow to the inflatable reservoir 30.

The dry powder formulation may be provided in or added to the inflatable reservoir 30, or, in the embodiment wherein the inhaler contains a chamber between the compressible component and the inflatable reservoir as shown in FIG. 1, the dry powder formulation may be provided in or added to the chamber. Further, the dry powder formulation may be provided in or added to the compressible component. In any of these embodiments, the air pulse discharged from the human-powered compressible component forms an aerosol of the dry powder formulation upon contact therewith. The powder, or a liquid aliquot, if a wet mist aerosol is desired, can be stored in and dispensed from a blister, capsule, mesh bag, or other dispensing device/container. In the embodiment of FIG. 1, the dry powder is shown at 60, arranged downstream of valve 42.

As shown in FIG. 1, the inflatable reservoir 30 includes an outlet valve 32, and the receiving mask 50 is provided in communication with the outlet valve 32. The receiving mask 50 is operable to receive an aerosol of dry powder from the inflatable reservoir 30, or from the compressible component directly in the embodiment in which the inflatable reservoir and the chamber are both omitted. Alternatively, a bolus of a dry powder formulation may be located in the receiving mask whereupon an air pulse from the compressible component outlet valve is received in the mask to aerosolize the formulation and deliver the aerosol to the patient. The receiving mask is operable to receive the aerosol from the chamber in the embodiment in which the inflatable reservoir is omitted but the chamber is included, and to deliver the aerosol to at least a mouth or nose of a patient. The outlet valve 32 from the inflatable reservoir is preferably a one-way valve, formed of flaps or other known design, to prevent contamination of the inflatable reservoir from breath moisture, sneezing, coughing, sputum, or the like.

The receiving mask 50 may be in the form of facemask, mouth-piece, or nose-piece, or other form as desired. In the embodiment of FIG. 1, the receiving mask comprises a flexible portion 54 terminating in a frame 52 adapted to cover the mouth and/or nose of a patient. The receiving mask flexible portion 54 may be relatively small in volume, for example about 20 to about 50 cc. In one embodiment, the receiving mask includes an exit valve, preferably a one-way exit valve, to allow the exiting of exhaled breath to the ambient air before the next breath is taken. An optional filter can be inserted upstream or downstream of the exit valve to protect the adjacent atmosphere, for example, health care givers, from exposure to the pharmaceutical formulation, for example, vaccine, drug, viral, bacterial, or fungal aerosols, if such constitute a potential hazard. Alternatively, the mask can itself be made from a porous filter material, which will also act as a fail safe prevention against accidental suffocation. In this embodiment, an outlet filter in the one way exit valve opening to the atmosphere, or the exit valve itself, may be unnecessary. A suitable mask material comprises a HEPA filter material, although one of ordinary skill in the art will recognize other materials suitable for use therein. In an alternate embodiment, the receiving mask comprises the frame 52 adapted to cover the mouth and/or nose of a patient, and the frame is attached at an end of the inflatable reservoir 30.

An optional screen or mesh, made, for example, of Nylon fibers, other plastics, silk fibers, or metal fibers may be inserted in the flow stream of the inhaler, downstream of a point where the aerosol is formed. The screen or mesh may be suitably sized to further disperse the particles and/or to exclude agglomerates or other particles too large to be inhaled from the air stream. In one embodiment, the screen or mesh has openings of a size of about 200×200 microns or less, but larger than the diameter of particles desired for delivery. Similarly, one or more additional flow dispersing protrusions, for example a dispersion or impaction plate, may be provided downstream of the point where the aerosol is formed to further disperse the particles and/or to exclude agglomerates or other particles too large to be inhaled from the air stream.

In a further embodiment, a human-powered vibration generator may be inserted in the flow stream to vibrate dry powder particles in the inhaler and improve dispersion thereof. The vibration generator may be positioned at any location prior to delivery of the aerosol to a patient. In a specific embodiment, the vibration generator is a sound vibration generator. For example, a sound vibration generator may be in the form of a sound producing reed, horn or whistle. FIG. 3 shows a schematic diagram of one embodiment of a sound vibration generator in the form of a horn 70 including a vibrating reed 72, which may be suitably placed in the air flow path in the inhaler, either upstream or downstream of the aerosol formation point to generate sound vibration as a patient inhales or as the air pulse is generated. Alternatively, in yet another embodiment, the sound vibration generator may be affixed to an exterior wall of the inhaler to vibrate the inhaler wall. A sound vibration generator is advantageous in that it provides an audible signal of air flow and the vibration generator is suitably placed downstream of the location of aerosol formation to assist in aerosol dispersion.

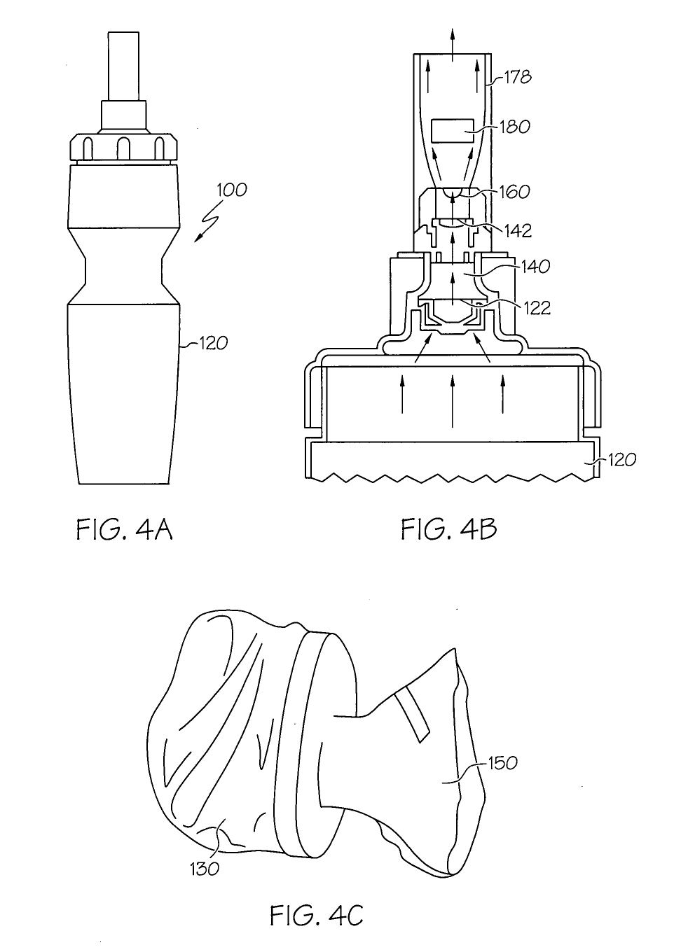

FIGS. 4A, 4B and 4C respectively show a plan view of one embodiment of the human-powered dry powder inhaler according to the invention (FIG. 4A), a schematic cross sectional view of the inhaler (FIG. 4B), and a view of a detachable inflatable reservoir and mask thereof with an aerosolized dose being delivered by the inhaler (FIG. 4C). With reference to FIGS. 4A-4C, the illustrated dry powder inhaler 100 includes a compressible component 120 in the form of a squeezable bottle, provided with a pressure release valve 122. A chamber 140 is arranged downstream of the valve 122 for throttling an air pulse discharged from the outlet valve 122 of the compressible component 120. A softer pressure release valve 142 is provided in the chamber. A bolus 160 of dry powder pharmaceutical formulation is arranged downstream of the valve 142. When the air pulse contacts the dry powder formulation, an aerosol of the dry powder is formed. The chamber expands at 178 to provide a connection for the inflatable reservoir. A dispersion plate 180 is provide in the flow path of the aerosol in order to further disperse the aerosol particles and/or to exclude agglomerates or other particles too large to be inhaled from the air stream. The distal end of the expansion 178 is adapted for connection to the inflatable reservoir 130 which is shown in FIG. 4C, together with the mouthpiece 150, to deliver the aerosol of the dry powder formulation to a patient.

The following examples illustrate embodiments according to the invention.

### EXAMPLE 1

The following formulations are formed into dry powders using the CAN-BD process:

TABLE 1
Formulations

| Formulation ID | Components |
|---|---|
| M50 | 50 g/L myo-inositol |
| M35man15 | 35 g/L myo-inositol, 15 g/L mannitol |
| M25man25 | 25 g/L myo-inositol, 25 g/L mannitol |
| M35S15 | 35 g/L myo-inositol, 15 g/L sorbitol |
| M50L2 | 50 g/L myo-inositol, 2 g/L leucine |
| M30G15 | 30 g/L myo-inositol, 15 g/L gelatin |

All of the above formulations also contain the following components: 25 g/L gelatin (except for M30G15), 16 g/L arginine-HCl, 1 g/L alanine, 2.1 g/L histidine, 3.5 g/L lactalbumin hydrolysate, 3 g/L tricine, pH 6.5-7.0

These dry powder formulations exhibit advantageous combinations of properties. For example, formulation M50 (50 g/L of myo-inositol) provides roughly spherical particles, as shown in FIG. 5, with slight dimpling also observed; the primary particle geometric diameter appears to be about 3 μm. Aerodynamic particle sizing confirms that most of the mass of the aerosolized particles is in the respirable size range (about 1-5 μm).

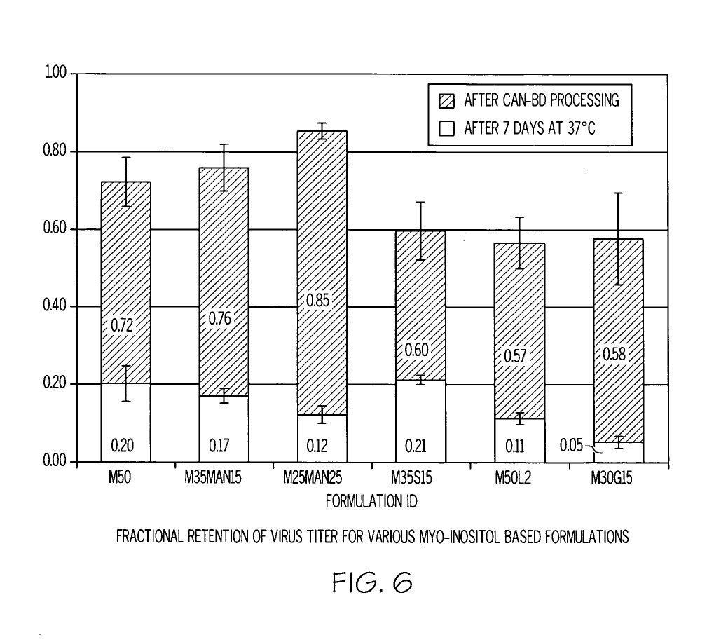

These formulations are desirable for use as a vehicle for syringe and needle-free delivery of vaccines in a dry powder inhaler according to the present invention. Live-attenuated measles vaccine virus powders are prepared using these formulations. Measles vaccine virus titers for the myo-inositol based formulations are measured and are set forth in FIG. 6. The M50 formulation shows a loss in virus titer after 7 days at 37° C. of only 0.6 log. For the M50 formulation, storage at 37° C. for 7 days does not cause a detectable decrease in fine particle fractions <5.8 μm and <3.3 μm when the powder was sufficiently protected from moisture ingress.

Formulations based on myo-inositol or myo-inositol combinations with mannitol, sorbitol, maltodextrin and/or other excipients are suitable for stabilizing the measles vaccine virus through CAN-BD processing and subsequent storage at 37° C. for 7 days. The addition of mannitol, sorbitol, maltodextrin or the like for part of the myo-inositol may be desirable to facilitate preparation of the dry powder due to the relatively low aqueous solubility of myo-inositol (140 g/L in pure water at 25° C., according to the Merck Index) compared to conventionally employed sugars and/or other materials, and/or to improve dispersibility. The formulations M50 and M50L2, which contain 25 g/L of gelatin as part of the formulation, display relatively low hygroscopicity. The sensitivity of powders to moisture uptake is important because the aerosol physical properties of inhalable dry powders are strongly dependent on moisture content: too much water can cause particle agglomeration, leading to reduced respirable fractions. The glass transition temperature of the dry formulations is also strongly dependent on water content: just a few percent increase in the water content of sugar based formulations can decrease the Tg by several tens of degrees Celsius. Higher moisture contents also result in decreased viral stability. Typical properties of the myo-inositol based formulations include: 1) FPF <5.8 μm and <3.3 μm of about 45-50% and about 20%, respectively; 2) onset and midpoint Tg of about 45 to 60° C. and 50 to 65° C., respectively; and 3) moisture contents of about 1% or less.

### EXAMPLE 2

Dry powder formulations of pure siRNA and of an equal part mixture of myo-inositol and siRNA are prepared from aqueous solutions using CAN-BD and a drying temperature of about 50° C. FIGS. 7A and 7B show scanning electron microscopy images of the dry powder formulations of, respectively, microparticles formed from pure siRNA in an aqueous solution (FIG. 7A) and microparticles formed from equal weights of myo-inositol and siRNA in an aqueous solution (FIG. 7B). The microparticles formed from equal weights of myo-inositol and siRNA exhibit more round and more uniform configurations. Additional improvements are obtained with the use of maltodextrin and/or lecithin in the formulations

The specific illustrations and embodiments described herein are exemplary only in nature and are not intended to be limiting of the invention defined by the claims. Further embodiments and examples will be apparent to one of ordinary skill in the art in view of this specification and are within the scope of the claimed invention.

What is claimed is:

1. A human-powered dry powder inhaler, comprising a human-powered compressible component operable to discharge an air pulse at an outlet at a pressure of about 1-40 psi; an inflatable reservoir operable to receive an air pulse discharged from the human-powered compressible component to provide an aerosol of a dry powder pharmaceutical formulation in the reservoir, the reservoir including an outlet valve; and a receiving mask in communication with the outlet valve and operable to receive an aerosol of dry powder from the reservoir and to deliver the aerosol to at least a mouth or nose of a patient.

2. The inhaler of claim 1, wherein the human-powered compressible component comprises a squeezable container having a pressure relief valve at the outlet.

3. The inhaler of claim 1, wherein the inflatable reservoir is formed of plastic or paper.

4. The inhaler of claim 1, wherein the human-powered compressible component is operable to discharge an air pulse at the outlet at a pressure of about 1-10 psi.

5. The inhaler of claim 4, wherein the human-powered compressible component is operable to discharge an air pulse at the outlet at a pressure of about 1-5 psi.

6. The inhaler of claim 4, wherein the human-powered compressible component is operable to discharge an air pulse at the outlet at a pressure of about 2 psi.

7. The inhaler of claim 1, wherein the inflatable reservoir outlet valve is a one-way valve which prevents flow from the receiving mask to the inflatable reservoir.

8. The inhaler of claim 1, wherein the inflatable reservoir includes a one-way inlet valve which prevents flow from the inflatable reservoir towards the compressible component.

9. The inhaler of claim 1, wherein the inflatable reservoir contains a dry powder pharmaceutical formulation.

10. The inhaler of claim 1, wherein a chamber is arranged between the outlet of the compressible component and the inflatable reservoir, wherein the chamber is operable to throttle an air pulse from the compressible component and discharge the throttled air pulse to the inflatable reservoir.

11. The inhaler of claim 10, wherein the chamber is operable to throttle an air pulse from the compressible component and discharge the throttled air pulse at an outlet at a pressure of less than about 2 psi to the inflatable reservoir.

12. The inhaler of claim 10, wherein a dry powder pharmaceutical formulation is provided in the chamber.

13. The inhaler of claim 10, wherein a dry powder pharmaceutical formulation is provided in the inflatable reservoir.

14. The inhaler of claim 1, wherein the receiving mask includes a one-way outlet valve operable to release an exhaled breath.

15. The inhaler of claim 14, wherein the one-way outlet valve includes a filter arranged to prevent release of dry powder pharmaceutical formulation to the atmosphere.

16. The inhaler of claim 1, wherein the receiving mask comprises a frame adapted to cover at least a mouth or nose of a patient.

17. The inhaler of claim 16, wherein the receiving mask comprises an inflatable portion terminating at the frame adapted to cover at least a mouth or nose of a patient.

18. The inhaler of claim 1, further comprising a filter or disperser arranged in the inflatable reservoir at a location operable to prevent delivery of dry powder agglomerates to a mouth or nose of a patient.

19. The inhaler of claim 1, further comprising a human-powered vibration generator.

20. A human-powered dry powder inhaler, comprising a human-powered compressible component operable to discharge an air pulse at an outlet of a polymeric pressure release valve at a pressure of about 1-40 psi; and a receiving mask in communication with the outlet of the compressible component and operable to deliver an aerosol of dry powder to at least a mouth or nose of a patient.

21. The inhaler of claim 20, wherein the human-powered compressible component is operable to discharge an air pulse at the outlet at a pressure of about 1-10 psi.

22. The inhaler of claim 21, wherein the human-powered compressible component is operable to discharge an air pulse at the outlet at a pressure of about 1-5 psi.

23. The inhaler of claim 22, wherein the human-powered compressible component is operable to discharge an air pulse at the outlet at a pressure of about 2 psi.

24. The inhaler of claim 20, further comprising a dry powder pharmaceutical formulation arranged downstream of the outlet of the compressible component, the dry powder pharmaceutical formulation comprising an active ingredient and a carrier.

25. The inhaler of claim 24, wherein the carrier comprises myo-inositol and/or maltodextrin and the dry powder pharmaceutical formulation comprises not more than about 5 weight percent water.

26. The inhaler of claim 24, wherein the carrier comprises myo-inositol and the dry powder pharmaceutical formulation comprises not more than about 1 weight percent water.

27. The inhaler of claim 24, wherein the dry powder pharmaceutical formulation comprises a vaccine.

28. The inhaler of claim 27, wherein the vaccine comprises measles virus.

29. The inhaler of claim 28, wherein the dry powder pharmaceutical formulation has a fine particle fraction of 50% less than 6 μm.

30. The inhaler of claim 28, wherein the dry powder pharmaceutical formulation has less than 1 log loss of viral activity of the vaccine upon incubation at 37° C. for 7 days.

31. The inhaler of claim 24, further comprising an inflatable reservoir operable to receive an air pulse discharged from the human-powered compressible component and to provide an aerosol of the dry powder pharmaceutical formulation in the inflatable reservoir, the inflatable reservoir including an outlet valve, wherein the receiving mask is in communication with the compressible component via the inflatable reservoir and is operable to receive an aerosol of dry powder from the inflatable reservoir and to deliver the aerosol to at least a mouth or nose of a patient.

32. The inhaler of claim 24, wherein a chamber is arranged between the outlet of the compressible component and the receiving mask, wherein the chamber is operable to throttle an air pulse from the compressible component and discharge the throttled air pulse to the receiving mask.

33. The inhaler of claim 32, wherein the chamber is operable to throttle an air pulse from the compressible component and discharge the throttled air pulse at an outlet at a pressure of less than about 2 psi to the receiving mask.

34. The inhaler of claim 20, wherein the receiving mask comprises an inflatable portion terminating in a frame adapted to cover at least a mouth or nose of a patient.

35. A method for delivery of a dry powder pharmaceutical formulation to a patient, comprising generating an air pulse at a pressure of about 1-40 psi using human power, using the air pulse to provide an aerosol of a dry powder pharmaceutical formulation in an inflatable reservoir, and delivering the resulting aerosol of dry powder pharmaceutical formulation to a receiving mask in communication with at least a mouth or nose of a patient, in the absence of electrical power and circuitry and pre-pressurized propellant gas.

36. The method of claim 35, wherein the dry powder pharmaceutical formulation comprises a vaccine, anti-viral, antibiotic or anti-inflammatory active ingredient.

37. The method of claim 35, wherein the dry powder pharmaceutical formulation further comprises a carrier, and wherein the carrier comprises myo-inositol and/or maltodextrin and the dry powder pharmaceutical formulation comprises not more than about 5 weight percent water.

38. The method of claim 37, wherein the active ingredient comprises a vaccine.

39. A method for delivery of a dry powder pharmaceutical formulation to a patient, comprising generating an air pulse at an outlet of a polymeric pressure release valve at a pressure of about 1-40 psi using human power, using the air pulse to aerosolize a dry powder pharmaceutical formulation, and delivering the resulting aerosol of dry powder pharmaceutical formulation to a receiving mask in communication with at least a mouth or nose of a patient, in the absence of electrical power and circuitry and pre-pressurized propellant gas.

40. The method of claim 39, wherein the aerosol of a dry powder pharmaceutical formulation is formed in an inflatable reservoir, and the inflatable reservoir is in communication with the receiving mask.

41. The method of claim 39, wherein the pharmaceutical formulation comprises a vaccine, anti-viral, antibiotic or anti-inflammatory active ingredient.

42. The method of claim 39, wherein the dry powder pharmaceutical formulation further comprises a carrier, and wherein the carrier comprises myo-inositol and/or maltodextrin and the dry powder pharmaceutical formulation comprises not more than about 5 weight percent water.

43. The method of claim 42, wherein the active ingredient comprises a vaccine.

44. A method for delivery of a dry powder pharmaceutical formulation to a patient, comprising generating an air pulse at a pressure of about 1-40 psi using human power, using the air pulse to aerosolize a dry powder pharmaceutical formulation, and delivering the resulting aerosol of dry powder pharmaceutical formulation to a receiving mask in communication with at least a mouth or nose of a patient, in the absence of electrical power and circuitry and pre-pressurized propellant gas, wherein the dry powder pharmaceutical formulation comprises an active ingredient and a carrier, wherein the carrier comprises myo-inositol and/or maltodextrin and the dry powder pharmaceutical formulation comprises not more than about 5 weight percent water.

45. The method of claim 44, wherein the dry powder pharmaceutical formulation comprises a vaccine.

46. The method of claim 45, wherein the vaccine comprises measles virus.

47. The method of claim 45, wherein the dry powder pharmaceutical formulation has a fine particle fraction of 50% less than 6 μm.

48. The method of claim 45, wherein the dry powder pharmaceutical formulation has less than 1 log loss of viral activity of the vaccine upon incubation at 37° C. for 7 days.

49. A dry powder pharmaceutical formulation comprising an active ingredient and a carrier, wherein the carrier comprises myo-inositol and/or maltodextrin and the dry powder pharmaceutical formulation comprises not more than about 5 weight percent water.

50. The dry powder pharmaceutical formulation of claim 49, wherein the active ingredient comprises a vaccine.

51. The dry powder pharmaceutical formulation of claim 50, wherein the vaccine comprises measles virus.

52. The dry powder pharmaceutical formulation of claim 50, wherein the dry powder pharmaceutical formulation has a fine particle fraction of 50% less than 6 μm.

53. The dry powder pharmaceutical formulation of claim 50, wherein the dry powder pharmaceutical formulation has less than 1 log loss of viral activity of the vaccine upon incubation at 37° C. for 7 days.

54. The dry powder pharmaceutical formulation of claim 49, wherein the active ingredient comprises small interfering RNA (siRNA).
